(19) United States
(12) Patent Application Publication
(10) Pub. No.: US 2022/0135454 A1
(43) Pub. Date: May 5, 2022

(54) A COMPOSITE MATERIAL AND A METHOD TO PREPARE THE COMPOSITE

(71) Applicants: CSIR, Pretoria (ZA); Mintek, Randburg (ZA)

(72) Inventors: Lucky SIKHWIVHILU, Johannesburg (ZA); Thembela HILLIE, Pretoria (ZA)

(21) Appl. No.: 17/430,078

(22) PCT Filed: Feb. 13, 2020
(86) PCT No.: PCT/IB2020/051169

§ 371 Date: Aug. 11, 2021

(30) Foreign Application Priority Data
Feb. 13, 2019 (ZA) 2019/00915

(51) Int. Cl. C02F 1/72; C02F 1/32; B01J 23/50; B01J 21/06; B01J 35/00; B01J 35/02; B01J 37/02; A61L 2/08; A61L 2/10

(52) U.S. Cl. CPC C02F 1/725; C02F 1/32; B01J 23/50; B01J 21/063; B01J 35/0013; B01J 35/004; A61L 2101/02; B01J 37/0215; A61L 2/088; A61L 2/10; C02F 2305/10; C02F 2303/04; C02F 2305/08; B01J 35/023

(57) ABSTRACT

The invention relates to a composite material, suitable for treating water. The composite material comprises an active layer and a substrate layer, with the active layer including titanium dioxide and silver nanoparticles. The active layer is bonded to the substrate layer such that, in use, there is substantially no leaching of the active layer into the body of water. The invention further relates to a method of preparing the composite material.

### INTRODUCTION

This invention relates to a composite material, comprising an active layer and a substrate layer, and a method to prepare the composite material. The invention further relates to the use of the composite material for reducing contaminants in water.

### BACKGROUND

Contaminated water, containing unacceptable levels of toxic substances and pathogens, is often used by humans for consumption, agricultural irrigation, or recreation. The use of such contaminated water leads to infection and development of disease. The World Health Organization (WHO) recommends that any water intended for drinking should contain zero total faecal coliform counts in any 100 ml sample. Due to poverty and scarcity of resources, the use of contaminated water, not complying to the WHO recommended standard, is still frequently used in many developing countries.

Currently, a number of methods are used for purifying water, including ultraviolet light, low frequency ultrasonic irradiation, distillation, reverse osmosis, water sediment filters, activated carbon, and ozonisation. A disadvantage of these methods is that they require relatively expensive apparatus, placing them out of reach of most of the population exposed to contaminated water.

Furthermore, halogens such as chlorine (CI) and bromine (Br) are well known and widely used as antibacterial agents, but the direct use of these halogens as antibacterial agents has, as a result of their high toxicity and high vapour pressure in pure form, many disadvantages. Another disadvantage associated with the use of halogens is that they may potentially react with other chemical contaminants to form by-products that could be carcinogenic.

Yet another disadvantage of these methods is that some of them, such as those using activated carbon, insufficiently remove contaminants including bacteria, algae and fungi from water. In addition, they may require an extended period of time to sufficiently reduce the contaminants. Another disadvantage experienced with some of the above methods are that they have limited efficiency whilst being costly and time consuming.

United States patent application no. US 2010/0032353 discloses a liquid dispenser or water purification unit having a housing and a mouthpiece configured for contact with the mouth of a person. At least part of the mouthpiece is provided with an antimicrobial surface coating comprising silver nanoparticles complexed with titanium dioxide. A disadvantage associated with US 2010/0032353 is that the filter embedded in the unit lasts only a year, (approximately 700 liters) meaning users must replace the unit annually. A further disadvantage is that the antimicrobial surface is not used for water purification, but to ensure that bacteria from one person holding or drinking from the mouthpiece are killed on contact with the antimicrobial surface, such that a second person using the mouthpiece is not infected by the bacteria. A device according to this invention will not be suitable for decontaminating a body of water.

International patent application publication no. WO 03/092886 discloses a mesoporous TiO₂ thin film, wherein the thin film is prepared from a TiO₂ sol-gel prepared from hydrolysis and condensation of titanium alkoxide in the presence of a stabilizer. A so-called dip coating technique is employed to create a TiO₂ thin film on the substrate. One particular disadvantage associated with the method disclosed for producing the coating on the substrate, is that the bond between the substrate and the nanoparticles cannot be sufficient in ensuring that nanomaterial particles are not leached into water on extended exposure thereto.

International patent application publication no. WO 2013/093805 discloses a method for preparing a nanotubular composite matrix for reducing contaminants in water, the method including the steps of preparing a titanium dioxide in nanotubular form, dissolving silver nanoparticles in a solvent, impregnating the titanium dioxide with the silver nanoparticles in solution to form a matrix, removing excess solvent from the matrix, and calcining the matrix to form a nanotubular composite matrix. The invention further relates to a method for reducing contaminants in water by incubating water exposed to the composite matrix and activating the matrix by exposure to a light source.

However, the disclosure fails to disclose a device or a method that can be used to decontaminate water without the nanomaterial particles being leached into the water. The disclosure further fails to disclose a method, whereby the composite matrix can be applied to a substrate such that, in use, the nanomaterials will not leach into the water being treated, whilst simultaneously maintaining efficiency comparable to that of the nanomaterial precursor material.

Therefore, there remains a need for a robust and effective method to produce a composite material, or a device comprising a composite material, that will ensure that the efficiency of the precursor material is maintained once bonded to the substrate, whilst simultaneously addressing the shortcomings of the prior art in terms of the leaching of the materials into the water being treated.

### SUMMARY OF THE INVENTION

According to a first aspect to the present invention there is provided a composite material suitable for treating a body of water, comprising an active layer and a substrate layer, wherein the active layer comprises titanium dioxide nanoparticles and silver nanoparticles, and wherein the active layer is deposited onto and bonded to the substrate layer such that, in use, there is substantially no leaching of the active layer into the body of water.

In one embodiment, the active layer has an average thickness of from about 25 nm to about 250 nm, preferably from about 50 nm to about 150 nm, more preferably from about 60 nm to about 85 nm.

In a preferred embodiment, the titanium dioxide nanoparticles are titanium dioxide nanotubes.

Preferably, titanium dioxide nanotubes have a cross-sectional diameter of from about 2 nm to about 200 nm, more preferably from about 5 nm to about 100 nm, and most preferably from about 8 nm to about 20 nm.

In one embodiment, the silver nanoparticles have a cross-sectional diameter of from about 0.5 nm to about 30 nm, preferably from about 1 nm to about 10 nm, more preferably from about 2 nm to about 5 nm.

According to a second aspect to the present invention there is provided a method for preparing a composite material comprising an active layer and a substrate layer, the method comprising providing an active layer solution of silver nanoparticle loaded titanium dioxide nanoparticles, aerosolizing the solution and directing the aerosol towards a heated substrate, thereby to deposit the active layer onto the substrate.

In one embodiment, the method comprises directing the aerosol through a nozzle towards the heated substrate, thereby to produce a coating spray.

In a preferred embodiment, the active layer solution of silver nanoparticle loaded titanium dioxide nanoparticles is aerosolized by placing the solution in a nebulizer bowl and flowing compressed air through the solution, thereby to form an aerosolized solution in one embodiment, the substrate was heated to a temperature of from about 100° C. to about 500° C., preferably from about 150° C. to about 300° C., more preferably to a temperate of about 250° C.

Preferably, the active layer solution is an aqueous solution.

Preferably, the active layer has an average thickness of from about 25 nm to about 250 nm, more preferably from about 50 nm to about 150 nm, and most preferably from about 60 nm to about 85 nm.

In a preferred embodiment, the active layer is deposited and bonded to the substrate such that, when in use in water, the active layer of the composite has a substantially zero leaching rate.

In one embodiment, the active layer solution of silver nanoparticle loaded titanium dioxide nanoparticles has a concentration of Ag/TiO₂ of from about 1% to about 10%, more preferably from about 2% to about 8%, most preferably about 5%.

According to a third aspect of the present invention there is provided a composite material produced according to the method of the invention.

According to a further aspect of the present invention there is provided a device suitable for use in disinfecting a body of the water, the device comprising a composite material according to the invention.

According to a further aspect to the present invention there is provided a method of disinfecting a body of water, the method comprising contacting a body of water with a composite material according to the invention, and exposing the water and composite material to a source of UV irradiation.

### BRIEF DESCRIPTION OF THE DRAWINGS

The invention will now be described in more detail with reference to the following non-limiting embodiments and figures in which:

FIG. 1 shows TEM images at 100 nm (a) and 20 nm (b) scale for the Ag/TiO₂ nanocomposite powder;

FIG. 2 shows a SEM surface morphology images of Ag/TiO₂ active layers produced using the method of the invention at (a) 150° C., (b) at 250° C., (c) at 350° C., and (d) at 450° C.;

FIG. 3 shows Ag/TiO₂ active layer thickness for active layers produced using the method of the invention at (a) 150° C., (b) at 250° C., (c) at 350° C., and (d) at 450° C.; and

FIG. 4 shows a graphical representation of the number if E. coli colonies detected, over time, in various water samples after treatment with the composite of the invention.

### DETAILED DESCRIPTION OF A PREFERRED EMBODIMENT

The present invention will now be described more fully hereinafter with reference to the accompanying figures, in which some of the non-limiting embodiments of the invention are shown.

The invention as described hereinafter should not be construed to be limited to the specific embodiments disclosed, with slight modifications and other embodiments intended to be included within the scope of the invention.

Although specific terms are employed herein, they are used in a generic and descriptive sense only and not for purposes of limitation.

As used herein, throughout this specification and in the claims which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having”, “including”, and variations thereof used herein, are meant to encompass the items listed thereafter, and equivalents thereof as well as additional items.

The present invention provides for a composite material that comprises an active layer and a substrate layer. The active layer comprises a nanocomposite matrix which includes titanium dioxide nanoparticles loaded with silver nanoparticles.

The titanium dioxide nanoparticles can be in the form of nanotubes, nanorods, or nanospheres. In a preferred embodiment according to the invention the titanium dioxide nanomaterial used in the preparation of the precursor matrix, and ultimately the active layer, is nanotubular titanium dioxide.

The silver loaded titanium dioxide nanocomposite is the precursor for the active layer. The precursor nanocomposite matrix is placed in solution and applied to a substrate as an active. It is of critical importance, according to an object of this invention, that the bonding at the interface between the active layer and the substrate be such that the nanomaterial leaching rate is such that is acceptable, and desirable, for extended exposure of the composite material to a body of water being treated in use. Furthermore, in terms of leaching of these materials into a substance to be consumed, it is not only the bonding between the active layer and the substrate that is important, but also the physical integrity of the active layer as such.

The active layer, comprising the silver loaded titanium dioxide nanomaterial, has to be applied to the substrate in such a manner that it provides a desirable bond and layer integrity, as described above. However, the primary function of the layer, i.e. the efficiency thereof in the process of removing contaminants from water, has to be maintained. This efficiency, compared to the efficiency of the nanocomposite precursor material, is integrally linked with maintaining the chemical structure, including the shape, size, and configuration, of the nanocomposite material once it is applied as a layer as part of a composite material.

The active layer, comprising the silver loaded titanium dioxide nanomaterial, is applied to the substrate at an average layer thickness, as measured using to scanning electron microscopy, of about 60 nm to about 85 nm. Preferably, active layer has an average layer thickness of about 25 nm to about 250 nm, about 25 nm to about 240 nm, about 25 nm to about 230 nm, about 25 nm to about 220 nm, about 25 nm to about 210 nm, about 25 nm to about 200 nm, about 25 nm to about 190 nm, about 25 nm to about 180 nm, about 25 nm to about 170 nm, about 25 nm to about 160 nm, about 25 nm to about 150 nm, about 25 nm to about 140 nm, about 25 nm to about 130 nm, about 25 nm to about 120 nm, about 25 nm to about 110 nm, about 25 nm to about 100 nm, about 25 nm to about 90 nm, and about 25 nm to about 85 nm.

Preferably, the active layer has an average layer thickness of about 30 nm to about 250 nm, about 30 nm to about 240 nm, about 30 nm to about 230 nm, about 30 nm to about 220 nm, about 30 nm to about 210 nm, about 30 nm to about 200 nm, about 30 nm to about 190 nm, about 30 nm to about 180 nm, about 30 nm to about 170 nm, about 30 nm to about 160 nm, about 30 nm to about 150 nm, about 30 nm to about 140 nm, about 30 nm to about 130 nm, about 30 nm to about 120 nm, about 30 nm to about 110 nm, about 30 nm to about 100 nm, about 30 nm to about 90 nm, and about 30 nm to about 85 nm.

More preferably, the active layer has an average layer thickness of about 40 nm to about 250 nm, about 40 nm to about 240 nm, about 40 nm to about 230 nm, about 40 nm to about 220 nm, about 40 nm to about 210 nm, about 40 nm to about 200 nm, about 40 nm to about 190 nm, about 40 nm to about 180 nm, about 40 nm to about 170 nm, about 40 nm to about 160 nm, about 40 nm to about 150 nm, about 40 nm to about 140 nm, about 40 nm to about 130 nm, about 40 nm to about 120 nm, about 40 nm to about 110 nm, about 40 nm to about 100 nm, about 40 nm to about 90 nm, and about 40 nm to about 85 nm.

Even more preferably, the active layer has an average layer thickness of about 50 nm to about 250 nm, about 50 nm to about 240 nm, about 50 nm to about 230 nm, about 50 nm to about 220 nm, about 50 nm to about 210 nm, about 50 nm to about 200 nm, about 50 nm to about 190 nm, about 50 nm to about 180 nm, about 50 nm to about 170 nm, about 50 nm to about 160 nm, about 50 nm to about 150 nm, about 50 nm to about 140 nm, about 50 nm to about 130 nm, about 50 nm to about 120 nm, about 50 nm to about 110 nm, about 50 nm to about 100 nm, about 50 nm to about 90 nm, and about 50 nm to about 85 nm.

Most preferably, the active layer has an average layer thickness of about 60 nm to about 250 nm, about 60 nm to about 240 nm, about 60 nm to about 230 nm, about 60 nm to about 220 nm, about 60 nm to about 210 nm, about 60 nm to about 200 nm, about 60 nm to about 190 nm, about 60 nm to about 180 nm, about 60 nm to about 170 nm, about 60 nm to about 160 nm, about 60 nm to about 150 nm, about 60 nm to about 140 nm, about 60 nm to about 130 nm, about 60 nm to about 120 nm, about 60 nm to about 110 nm, about 60 nm to about 100 nm, about 60 nm to about 90 nm, and about 60 nm to about 85 nm.

In one embodiment of the invention the active layer comprising silver nanoparticles loaded onto nanotubular titanium dioxide. The nanotubular titanium dioxide in the active layer, i.e. after application and bonding of the active layer to the substrate, have a cross-sectional diameter of about 2 nm to about 200 nm, about 5 nm to about 180 nm, about 5 nm to about 160 nm, about 5 nm to about 150 nm, about 5 nm to about 140 nm, about 5 nm to about 130 nm, about 5 nm to about 120, about 5 nm to about 110 nm, about 5 nm to about 100 nm, about 6 nm to about 90 nm, about 6 nm to about 80 nm, about 6 nm to about 70 nm, about 6 nm to about 60 nm, about 6 nm to about 50 nm, about 6 nm to about 40 nm, about 6 nm to about 30 nm, about 6 nm to about 20 nm, about 7 nm to about 20 nm, about 8 nm to about 20 nm, about 10 nm to about 20 nm.

The silver nanoparticles in the active layer, i.e. after application and bonding of the active layer to the substrate, have a cross-sectional diameter of from about 0.5 nm to about 30 nm. Preferably, the silver nanoparticles have a cross-sectional diameter from about 1 nm to about 10 nm, about 1 nm to about 8 nm, about 1 nm to about 7 nm, about 1 nm to about 6 nm about, and about 1 nm to about 2 nm. More preferably, the silver nanoparticles have a cross-sectional diameter of from about 2 nm to about 10 nm, about 2 nm to about 8 nm, about 2 nm to about 7 nm, about 2 nm to about 6 nm, and preferably from about 2 nm to about 5 nm.

The substrate may be any suitable substrate that can withstanding the temperatures to which the substrate will be exposed to during the coating process. Suitably substrates include, but are not limited to, glass, stainless steel, and ceramic material.

The present invention further provides for a method for preparing a composite material that comprises an active layer and a substrate layer, the method comprising providing an active layer solution of silver nanoparticle loaded titanium dioxide nanoparticles, aerosolizing the solution and directing the aerosol towards a heated substrate, thereby to deposit the active layer onto the substrate.

The active layer solution, comprising the silver loaded titanium dioxide nanoparticles, aerosolized by placing the solution in a nebulizer bowl and flowing compressed air through the solution, thereby to form an aerosolized solution. However, an aerosol of the solution may be produced in any other manner, including by ultrasonication.

As used in this specification, the term “aerosol”, “aerosolizing” and “aerosolized” should be understood to describe a mixture or a suspension of fine solid particles, and/or liquid particles, in air or any another gas.

The aerosolized solution is directed at the substrate to be coated through a spray nozzle, thereby creating an aerosol spray of the active layer solution. The substrate to be coated glass, stainless steel, or a ceramic type material. In one embodiment of the invention, the active layer was coated onto a glass substrate that was heated to a temperate of about 250° C.

While a temperature of 250° C. may be preferred for a particular type of glass, it will be understood that this temperature may vary depending on the substrate to be coated. In particular, the temperature of the substrate may be from about 100° C. to about 500° C. The temperature of the substrate may be from about 100° C. to about 400° C., about 100° C. to about 300° C., or about 100° C. to about 250° C.

The temperature of the substrate may be from about 150° C. to about 500° C. In particular, the temperature may be about 150° C. to about 400° C., about 150° C. to about 300° C., or about 150° C. to about 250° C., limits inclusive. For example, the temperature of the substrate may be about 150° C., about 160° C., about 170° C., about 180° C., about 190° C., about 200° C., about 210° C., about 220° C., about 230° C., about 240° C., or about 250° C.

The concentration of TiO₂ and Ag in the active layer solution may be about 1% to about 10%, about 2% to about 8%, about 3% to about 6%, or about 5%.

Preparation of TiO₂ Nanotubes, Nanorods or Nanospheres

The titanium dioxide nanoparticles were also prepared from a commercial P25 Degussa titania powder. In order to prepare the titanium dioxide nanotubes, about 23 g of the Degussa titania powder was placed in a teflon container together with 150 ml of 18 M potassium hydroxide to form a mixture.

The mixture was stirred at a constant rate of 500 rpm in an autoclave at a temperature of about 150° C. for 24 hours. Alternatively, the mixture was stirred at a constant rate of 500 rpm in a microwave reactor for about 5-10 minutes. The mixture was allowed to cool at room temperature for 24 hours to form titanium dioxide nanotubes.

Subsequently, the nanotubes were separated from the mixture by centrifugation at a stirring rate of 10 000 rpm for 30 minutes at about 4° C. The nanotubes were again washed with deionised water to remove any excess potassium hydroxide from the nanotubes thereby to maintain conductivity of the nanotubes constant below 100 pS/cm.

The nanotubes were dried in an oven at about 120° C. for 12 hours and then allowed to cool to room temperature for 5 hours. The nanotubes were then sieved to a desired size. The nanotubes had a cross-sectional diameter of from about 7 nm to about 11 nm. However, the nanotubes may also have a cross-section diameter of about 2 nm to about 200 nm, about 5 nm to about 180 nm, about 5 nm to about 160 nm, about 5 nm to about 150 nm, about 5 nm to about 140 nm, about 5 nm to about 130 nm, about 5 nm to about 120, about 5 nm to about 110 nm, about 5 nm to about 100 nm, about 6 nm to about 90 nm, about 6 nm to about 80 nm, about 6 nm to about 70 nm, about 6 nm to about 60 nm, about 6 nm to about 50 nm, about 6 nm to about 40 nm, about 6 nm to about 30 nm, about 6 nm to about 20 nm, about 7 nm to about 20 nm, about 8 nm to about 20 nm, about 10 nm to about 20 nm.

The above method as described was also substantially followed for the preparation of the titanium dioxide in a nanorods form, with the exception that 10 M of potassium hydroxide was used.

The titanium dioxide in a nanospherical form was prepared by using the above commercial P25 Degussa titania powder. The powder was modified prior to use. The modification was achieved by mixing the powder with water and then compressing the mixture into a moist paste. This moist paste was then dried in an oven at 120° C. for 48 hours to form the titanium dioxide nanospheres. The nanospheres are cooled and crushed into fragments. The fragments having the desired size of the nanospherical form are sieved out. The larger fragments are recycled and re-sieved while the smaller ones (fine powder formed as a result of crushing) are mixed with water again to from the moist paste to be dried.

Loading Titanium Dioxide Nanoparticles with Silver

Silver nitrate was prepared and loaded onto the titanium dioxide nanotubes, nanorods or nanospheres. About 5% w/w of the silver nitrate was dissolved in 10 ml of deionised water to form a mixture. The mixture was then added to a container containing about 4 g of the nanotubes, nanorods or nanospheres to form a matrix.

Excess water was removed by drying the matrix at room temperature for 24 hours and subsequently in an oven at 100° C. for a further period of 12 hours. It was observed that the concentration of the loaded silver nitrate differs based on the nature of the TiO₂ form used. In all instances, nitrate was used as a precursor, and water was used as a solvent due to excellent solubility of the nitrate in water. The matrix was calcined at 200° C. for 5 hours to remove the nitrate moiety from the silver nitrate to form a composite matrix (nanotubular, nanorod, or nanospherical) material to be uses as a precursor in the formation of the active layer.

Synthesis of Composite Material Comprising Ag/TiO₂ Nanocomposite Active Layer

In the method according to the present invention, the Ag/TiO₂ nanocomposite, prepared according to the method described above, was placed into solution, which solution was aerosolized and sprayed through a coating nozzle onto a heated surface.

The apparatus that was used for aerosolizing and spraying the solution is comprises a spray unit, a liquid feeding unit, and a temperature control unit (Carbolite furnace). The spray unit was similar to that used by asthma patients in the delivery of pharmaceutical products. The spray unit consists of a compressor, a nebulizer bowl, and tubing.

To achieve deposition, the Ag/TiO₂ nanocomposite solution was placed in the nebulizer bowl, with the bowl and the compressor being connected through the required tubing. The compressor provides compressed air that compresses the liquid precursor into a fine mist or aerosol. The aerosolized active layer solution, comprising the Ag/TiO₂ nanoparticles, flows through the tubing towards the coating nozzle via a globe valve, which is used to control the flow of the aerosol to the heated substrate. The aerosolized active layer solution flows across the tubing to the furnace, where the glass substrate to be coated was placed inside a glass tube.

In order to obtain optimal characteristics of the active layer, parameters such as substrate temperature, layer thickness and other deposition conditions were investigated. To optimize the synthesis of the active layer, in terms of bonding and maintaining the characteristics of the composite matrix precursor material, substrate temperatures were varied from 150° C. to 450° C. (150° C., 250° C., 350° C. and 450° C.). Application time of the aerosol spray to the substrate was set at 1 hour. It was found that the active layer thickness depends, in part, on the position of the substrate in the furnace.

Active Layer Characterisation

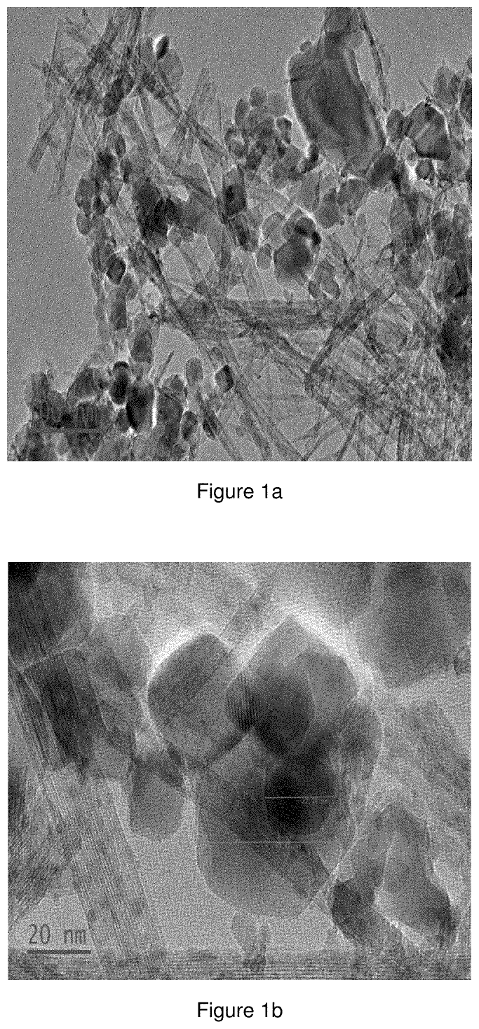

Transmission Electron Microscopy (TEM) was performed using a JEOL 2010 high-resolution transmission electron microscope with a LaB₆— cathode operated at a voltage of 200 kV on the Ag/TiO₂ nanocomposite powder. As can be seen from the TEM images in FIG. 1, both image (a) and (b) shows the distribution of silver nanoparticles and titanium dioxide nanotubes. The TEM images also show that silver nanoparticles are found on the surface of titanium dioxide nanotubes (FIG. 1b, scale 20 nm). The silver nanoparticles found on the surface of the titanium dioxide nanotubes has an average diameter of about 2-5 nm. Further, as can be seen from FIG. 1, the titanium dioxide nanotubes were needle-shaped with an average cross-sectional diameter ranging from about 8-20 nm.

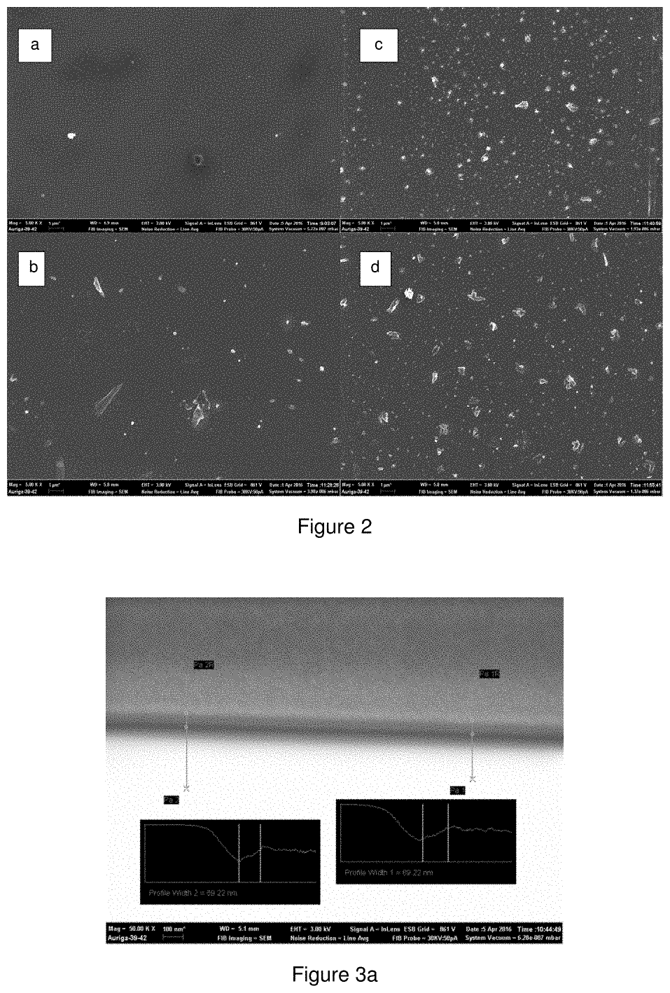

FIG. 2 shows scanning electron microscopy (SEM) images of the active layers produced at various temperatures. The surface morphology of the Ag/TiO₂ nanomaterial containing active layers were investigated by Joel SEM (JSM-5800 LV, JEOL) at 5000× magnification.

During deposition, the surface energy of the glass substrate is higher than the surface tension of the solution. Droplets tend to blast on the glass substrate creating a uniform deposition of particles. This process may be referred to as wetting. As the thickness increases, surface energy tends to be lower than the surface tension on top of the deposited active layer. This leads to the formation of particles on top of the active layer.

FIG. 2a shows the active layer deposited at 150° C., and it can be seen that the layer's surface consists of small and non-uniform crystallites. The surface has a sparsity of particles. FIG. 2b shows the active layer deposited at 250° C. It can be observed that particles in this temperature start to adhere on top of the layer when the solution is allowed to evaporate and deposit onto the layer, thickness tends to increase. As the temperature increases from 250° C. to 350° C., and to 450° C., as is shown in FIGS. 2b-2d respectively, more particles are observed with varying particle size and shape.

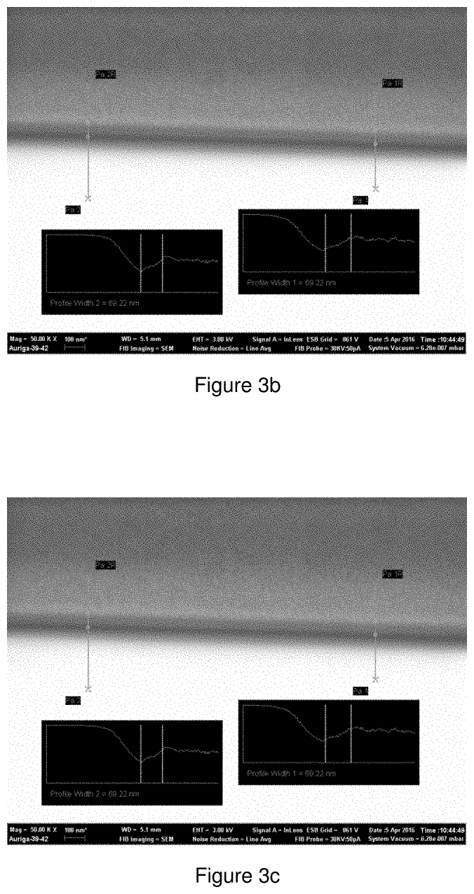

In order to confirm that an active layer was produced, thickness was measured with JEOL SEM (JSM-5800 LV, JEOL) using a cross sectional method. As can be seen in FIG. 3, the average active layer thickness increased linearly with increasing substrate temperature. Without thereby wishing to be bound by any particular theory, it is belied that, during aerosol spray deposition, droplets reach the heated substrates and undergo adhesion, suggesting that at higher temperatures (250° C., 350° C. and 450° C.) the rate of evaporation of the solution is much faster, which results in an increase inlayer thickness. This can also be seen from FIG. 2 that particles on the surface of the layer at high temperatures become uniform. The active layer produced at a temperature of 150° C. (FIG. 2a), which has particles sparsely on its surface morphology, has an average thickness of 62.2 nm (FIG. 3a).

The active layer produced at 250° C., shown in FIG. 2b, has an average thickness of 73.69 nm (FIG. 3b). Compared to the film produced at 150° C., it can be seen that the temperature has an influence on the active layer thickness. In FIG. 3c, the active layer has an average thickness of 82.62 nm, while in FIG. 3d the active layer also has an average thickness of 82.62 nm. This would appear to suggest that as the temperature increases, the active layer thickness increases, but only up to a certain optimised temperature point. Since the surface energy of the substrate becomes lower than the surface tension of the droplets, thickness tends to be constant but the formation of particulates tends to increase. This can be seen in FIGS. 3c and 3d respectively.

Efficacy Studies—Antibacterial Tests

Escherichia coli (E. coli, ATCC 15597) and salmonella was selected for antibacterial testing of the composite material of the invention. Three water sample types, namely river water, borehole water and tap water, were filtered and tested before dosing with known amounts of E. coli and salmonella, thereby to determine the number of colonies present.

Three water samples were dosed with E. coli and salmonella then filtered to determine the number of E. coli and salmonella colonies present. This was done for a comparison of the number of colonies present prior to and after treatment.

After dosing with E. coli (2.5 pL of E. coli ATCC15597), water samples (12 ml each) were left to stand in the sun with a composite material according to the invention, produced at 250° C., added to each sample. The samples were left to stand in the sun for 15 minutes, 30 minutes, and 60 minutes respectively. Three samples were tested for each water type. After exposure, the samples were tested for the number of E. coli colonies. A similar process was followed for salmonella testing.

The method used to measure microbial count was the Heterotropic Count Microbial Indicator method (as described in Bortram et al., 2003). This method was used to monitor the performance disinfection process.

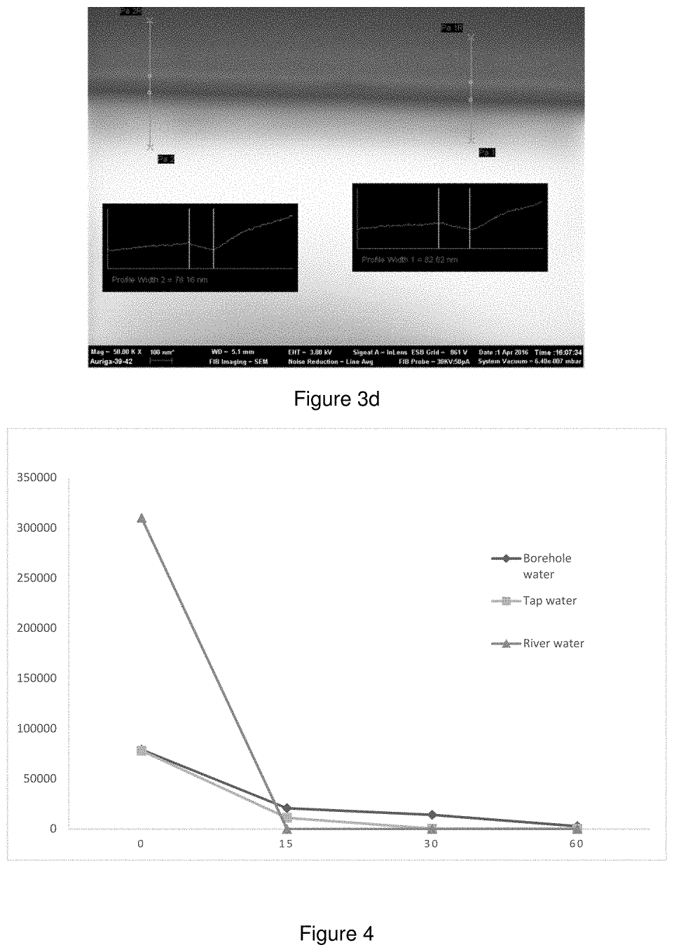

Referring now to FIG. 4, on average, after exposure of 15 minutes to the composite material of the invention a reduction of 74%, 86%, and 99% were seen for the borehole water, tap water and river water samples, respectively. After 30 minutes of exposure, a 99% reduction was seen for tap water, and after 60 minutes an 81% reduction for borehole water samples.

In addition to the above efficiency tests, a cycle test was conducted to determine whether the active layer according to the present invention would maintain its efficiency over an extended period of time. During the cycle test, 2 L of each water samples were initially tested to determine the level of E. coli present. The water samples (borehole, tap and river water) were dosed with 2.5 pL of E. coli ATCC15597, after which samples were split into 5 separate 120 ml samples and labelled “Cycle 1” to “Cycle 5”.

Composite material comprising an active layer of silver loaded titanium dioxide nanomaterial, produced at 250° C., was first inserted into each water sample labelled Cycle 1, and left for 60 minutes. After 60 minutes, composite material was transferred to the next sample labelled Cycle 2 and left for 60 minutes. This procedure was repeated up to the last sample, labelled Cycle 5, for each of the three water sample types. Results obtained for the different water types, dosed with E. coli, are shown in Table 1 below.

TABLE 1
Number of E. coli colonies present after 60
minutes per cycle for various water samples.

| Water sample | After spiking | Cycle 1 | Cycle 2 | Cycle 3 | Cycle 4 | Cycle 5 |
|---|---|---|---|---|---|---|
| Tap | 470 000 | <1 | <1 | <1 | <1 | <1 |
| Borehole | 420 000 | 2 000 | 110 | 14 | <1 | <1 |

This above description of some of the illustrative embodiments of the invention is to indicate how the invention can be made and carried out. Those of ordinary skill in the art will know that various details may be modified thereby arriving at further embodiments, but that many of these embodiments will remain within the scope of the invention.

1. A composite material suitable for treating a body of water, comprising an active layer and a substrate layer, wherein the active layer comprises titanium dioxide nanotubes loaded with silver nanoparticles, and wherein the active layer is deposited onto and bonded to the substrate layer such that, in use, there is substantially no leaching of the active layer into the body of water.

2. The composite material according to claim 1, wherein the active layer has an average thickness of from about 25 nm to about 250 nm, preferably from about 50 nm to about 150 nm, more preferably from about 60 nm to about 85 nm.

3. The composite material according to claim 1, wherein the titanium dioxide nanotubes in the active layer, after application and bonding of the active layer to the substrate, have a cross-sectional diameter of from about 2 nm to about 200 nm, more preferably from about 5 nm to about 100 nm, and most preferably from about 8 nm to about 20 nm.

4. The composite material according to claim 1, wherein the loaded silver nanoparticles in the active layer, after application and bonding of the active layer to the substrate, have a cross-sectional diameter of from about 0.5 nm to about 30 nm, preferably from about 1 nm to about 10 nm, more preferably from about 2 nm to about 5 nm.

5. A method for preparing a composite material comprising an active layer and a substrate layer, the method comprising providing an active layer solution of silver nanoparticle loaded titanium dioxide nanotubes, aerosolizing the active layer solution and directing the aerosol towards a heated substrate, thereby to deposit the active layer onto the substrate.

6. The method according to claim 5, wherein the method comprises directing the aerosol through a nozzle towards the heated substrate, thereby to produce a coating spray.

7. The method according to claim 5, wherein the active layer solution of silver nanoparticle loaded titanium dioxide nanotubes is aerosolized by placing the solution in a nebulizer bowl and flowing compressed air through the solution, thereby to form an aerosolized solution.

8. The method according to claim 5, wherein the substrate is heated to a temperature of from about 100° C. to about 500° C., preferably from about 150° C. to about 300° C., more preferably to a temperate of about 250° C.

9. The method according to claim 5, wherein the active layer solution is an aqueous solution.

10. The method according to claim 5, wherein the active layer has an average thickness of from about 25 nm to about 250 nm, more preferably from about 50 nm to about 150 nm, and most preferably from about 60 nm to about 85 nm.

11. The method according to claim 5, wherein the active layer is deposited and bonded to the substrate such that, when in use in water, the active layer of the composite has a substantially zero leaching rate.

12. The method according to claim 5, wherein the active layer solution of silver nanoparticle loaded titanium dioxide nanotubes has a concentration of Ag/TiO₂ of from about 1% to about 10%, more preferably from about 2% to about 8%, most preferably about 5%.

13. A composite material produced according to the method of claim 5.

14. A device suitable for use in a method of disinfecting a body of water, the device comprising a composite material according to claim 1.

15. A method of disinfecting a body of water, the method comprising contacting the body of water with a composite material according to claim 1, and exposing the water and composite material to a source of UV irradiation.

16. The composite material according to claim 1, wherein the active layer is deposited and bonded to the substrate such that, when in use in water, the active layer of the composite has a substantially zero leaching rate.
